(19) United States
(12) Patent Application Publication
(10) Pub. No.: US 2009/0247981 A1
(43) Pub. Date: Oct. 1, 2009

(54) TAMPON PACKAGE AND METHOD FOR MAKING THE SAME

(75) Inventors: Frank S. Glaug, Chester Springs, PA (US); Keith J. Edgett, Middletown, DE (US); Eugene P. Dougherty, JR., Camden-Wyoming, DE (US); Douglas L. Tackett, Dover, DE (US)

Correspondence Address: MICHAUD-DUFFY GROUP LLP, 306 INDUSTRIAL PARK ROAD, SUITE 206, MIDDLETOWN, CT, 06457, US

(73) Assignee: Playtex Products, Inc., Westport, CT (US)

(21) Appl. No.: 12/057,510

(22) Filed: Mar. 28, 2008

(51) Int. Cl. A61F 13/20; A61B 19/02

(52) U.S. Cl. 604/385.02; 604/385.17; 206/440; 206/438

(57) ABSTRACT

A tampon package includes a tampon assembly sealed within packaging material. The packaging material is continuously sealed about its perimeter to form an internal air-tight chamber for retaining the tampon assembly therein. The tampon package includes a tampon pledget disposed within an applicator barrel. In one embodiment, the packaging material is made from a water-barrier material. The water-barrier material may be a laminate of polyolefin or polyethylene terephthalate and an ethylene vinyl acetate material.

### BACKGROUND OF THE INVENTION

1. Field of the Invention.

This invention relates generally to tampons packages and, more particularly, to tampon packages having a continuous perimeter seal enclosing a tampon assembly within an air, moisture and contaminate resistance wrapper.

2. Description of Related Art.

Generally speaking, a tampon pledget is commonly sold as part of a tampon assembly that includes the tampon pledget and an applicator in which the tampon pledget is disposed. The applicator typically includes an applicator barrel that retains the tampon pledget therein and a plunger for ejecting the tampon pledget from the applicator barrel. Tampon assemblies vary with regard to a force required for a user to eject the tampon pledget from the applicator barrel. However, the ejection force is generally intended to be relatively minimal to permit ease of use.

Tampon assemblies are commonly packaged individually in an overwrap (hereinafter simply “wrapper”). Conventional wrappers include paper and other materials that typically allow air, moisture and contaminates to penetrate the wrapper and contact the tampon assembly. As can be appreciated, tampon assemblies may be stored under various conditions and for various lengths of time prior to use subjecting the assemblies to “aging.” Aging can occur while tampons are in the commercial distribution chain and after purchase by consumers. It has been found that aging, particularly aging under humid ambient conditions, can cause “blooming.” Blooming is a condition where moisture is absorbed by absorbent materials of the tampon pledget causing the pledget to radially expand. As can be appreciated, it is undesirable for the tampon pledget to prematurely expand (e.g., bloom) within the applicator barrel. Premature expansion of the tampon pledget within the barrel results in an increase in ejection force needed to expel the at least partially expanded tampon pledget from the applicator barrel. Blooming continues as long as the moisture remains in contact with the tampon pledget. As such, a significant increase in the needed ejection force may result if a tampon pledget is exposed to moisture for a prolonged period of time. As can be appreciated, any significant increase in the ejection force needed to expel a tampon pledget from an applicator barrel is undesirable.

Accordingly, the inventors have recognized that a need exists for improved tampon packaging that can offset and, preferably, eliminate blooming of tampon pledgets within an applicator barrel. The inventors have discovered that tampon packaging having a continuous perimeter seal to enclose the tampon assembly within an air, moisture and contaminate resistance wrapper overcomes the aforementioned, perceived problems and drawbacks of premature tampon pledget blooming that can lead to an increase in the ejection force needed to expel the tampon pledget from the applicator barrel.

### SUMMARY OF THE INVENTION

The present invention resides in one aspect in a tampon package including a tampon assembly enclosed within a package material. The tampon assembly includes a tampon pledget enclosed within an applicator barrel. The packaging material has a perimeter, and is continuously sealed about the perimeter to form an internal air-tight chamber for retaining the tampon assembly therein.

In one embodiment, the packaging material is comprised of a water-barrier material. In one aspect of the invention, the water-barrier material may be comprised of a laminate containing a layer of polyolefin or polyethylene terephthalate (PET) base and a layer comprising a vinyl acetate sealant. For example, the water-barrier material may comprise a layer of polyethylene (PE) base and a layer of ethylene vinyl acetate (EVA) sealant and, optionally, the EVA may be inside of the overwrap.

In another embodiment, the water barrier material may comprise a layer of polypropylene (PP) base and a layer of ethylene vinyl acetate (EVA) sealant. In yet another embodiment, the water barrier material may comprise a layer of polyethylene (PE) base and a layer of high density polyethylene (HDPE) sealant. In still another embodiment, the water barrier material may comprise a layer of polyethylene (PE) base and a layer of polypropylene (PP) sealant.

### BRIEF DESCRIPTION OF THE DRAWINGS

The features and advantages of the present invention will be better understood when the Detailed Description of the Preferred Embodiments given below is considered in conjunction with the figures provided.

FIG. 1 is a schematic cross-sectional view of a tampon assembly in a C-folded sheet of water barrier material as described herein.

FIG. 2 is a schematic plan view of a tampon assembly package comprising a wrapper as described herein.

FIG. 3 is a schematic plan view of another tampon assembly package comprising a wrapper as described herein.

FIG. 4 is a schematic plan view of a portion of yet another tampon assembly package comprising a wrapper as described herein.

FIG. 5 illustrates experimental results depicting adverse affects of aging under humid conditions has upon conventional tampon assemblies.

In these figures like structures are assigned like reference numerals, but may not be referenced in the description of all figures.

### DETAILED DESCRIPTION OF THE PREFERRED EMBODIMENTS

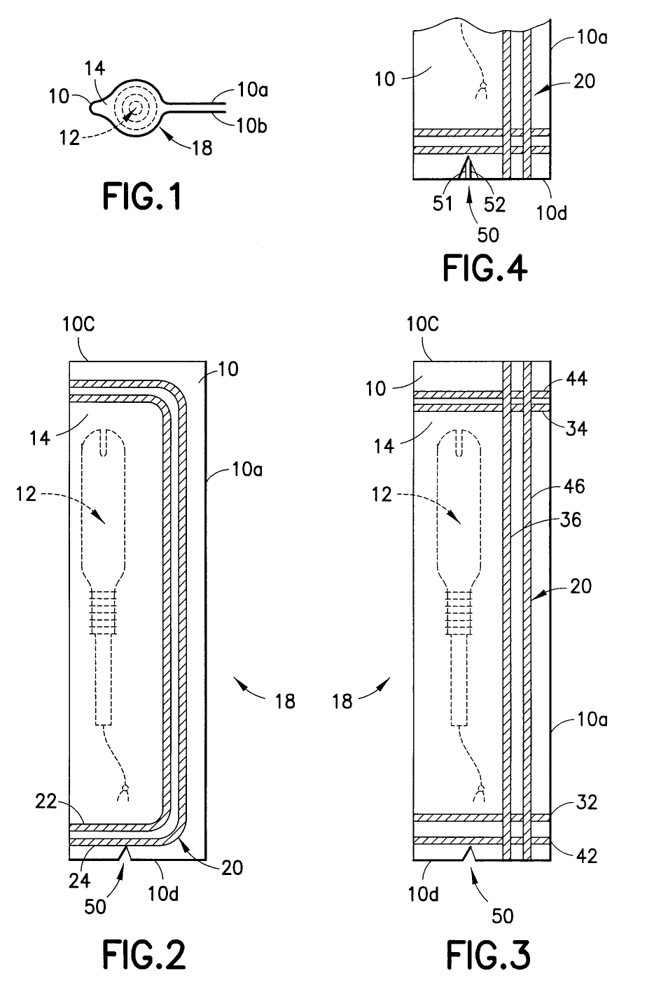

As illustrated in FIG. 1, a tampon assembly 12 is wrapped with a package material 10 that effectively surrounds the tampon assembly 12 and encloses the assembly within an internal chamber 14. The package material 10 is sealed about its perimeter such that the internal chamber 14 is substantially air-tight to form, as described herein, an improved tampon package 18. In one embodiment, the package material 10 includes a sheet of thermoplastic material wrapped around the tampon assembly 12 in a C-type fold arrangement. As shown in FIGS. 2 and 3, the package material 10 is sealed with a continuous perimeter seal, shown generally at 20, such that a first side 10a of the material 10 is affixed to a second side 10b of the material 10.

In accordance with the present invention, the package material 10 is a sheet of thermoplastic material that forms a barrier to prevent air, moisture and contaminates from entering the internal chamber 14 and contacting the tampon assembly 12 enclosed therein. In one embodiment, the thermoplastic material is comprised of a single sheet of synthetic polymeric material and, optionally, a laminate comprising two or more layers of thermoplastic materials. In one embodiment, the package material 10 is comprised of a water-impermeable material such as, for example, a co-extruded laminate that comprises a polyolefin layer and a layer of a vinyl acetate material. In one embodiment, the co-extruded laminate includes a layer of polyethylene (PE) base and a layer of ethylene vinyl acetate (EVA) sealant. Alternatively, a layer of polyester such as, for example, polyethylene terephthalate (PET) or polypropylene (PP) may be used in place of the PE layer. However, the inventors have recognized that PE provides a wrapper that produces less noise during opening than a wrapper that is comprised of PET or PP. In one embodiment, a thickness of the package material 10 is in a range of about 0.6 to 3.6 mils, and preferably, in a range of about 1.0 to 1.6 mils. In one embodiment, a thickness of the sealant layer (e.g., the ethylene vinyl acetate (EVA) layer), is expressed in a percentage add-on and is in a range of about five to twenty-five percent (5-25%) and preferably, about nine to eighteen percent (9-18%).

In one embodiment, the package material 10 is heat sensitive such that the perimeter seal 20 is formed by, for example, a heat press, sonic welding, continuous bead hot melt adhesive, or any other suitable means for affixing the first side 10a to the second side 10b of the material 10. In one embodiment, the package material is comprised of a co-extruded laminate that includes layers of polypropylene (PP) and EVA, with the EVA layer disposed inside the PP layer of the wrapper. The co-extruded laminate of PP/EVA is available from Pliant Corporation (Newport News, VA) as product code XP9475A.

As shown in FIG. 1 and as described above, the improved tampon package 18 is formed by folding the sheet of thermoplastic material 10 in a C-type fold arrangement around the tampon assembly 12. The continuous perimeter seal 20 affixes two side portions 10a and 10b of the package material 10 together. As shown in FIGS. 2 and 3, the seal 20 includes at least one continuous seal (e.g., seal 22 and seals 32, 34, and 36) traversing the perimeter of the package material 10. In one embodiment, the continuous perimeter seal 20 also includes at least a second continuous seal. For example, as shown in FIG. 2, the continuous perimeter seal 20 includes a first unitary perimeter seal 22 formed in a single-step sealing operation such as by a sealing press or the like, and a second unitary perimeter seal 24 formed in a single-step sealing operation. It should be appreciated that the first seal 22 and the second seal 24 may be formed simultaneously or consecutively. As shown in FIG. 3, it is also within the scope of the present invention for the continuous perimeter seal 20 to be formed in a multi-step sealing process. For example, a first step of a sealing process employs a linear thermal press to form a first end seam 32 and a second end seam 34 at a first end 10c and a second end 10d of the package material 10, respectively. In a second step of the sealing process a side seam 36 is formed intersecting the first end seam 32 and the second end seam 34. As can be appreciated, the first end seam 32, the second end seam 34, and the side seam 36 cooperate to form the continuous perimeter seam 20 to seal the tampon assembly 18 within the internal chamber 14 of the improved tampon package 18. In one embodiment, at least a second group of a first end seam 42, a second end seam 44, and a side seam 46 are formed at the perimeter of the package material 10. It should also be appreciated that the end seals 32 and 34, and 42 and 44 may be formed simultaneously or consecutively. Similarly, the side seals 36 and 46 may be formed simultaneously or consecutively.

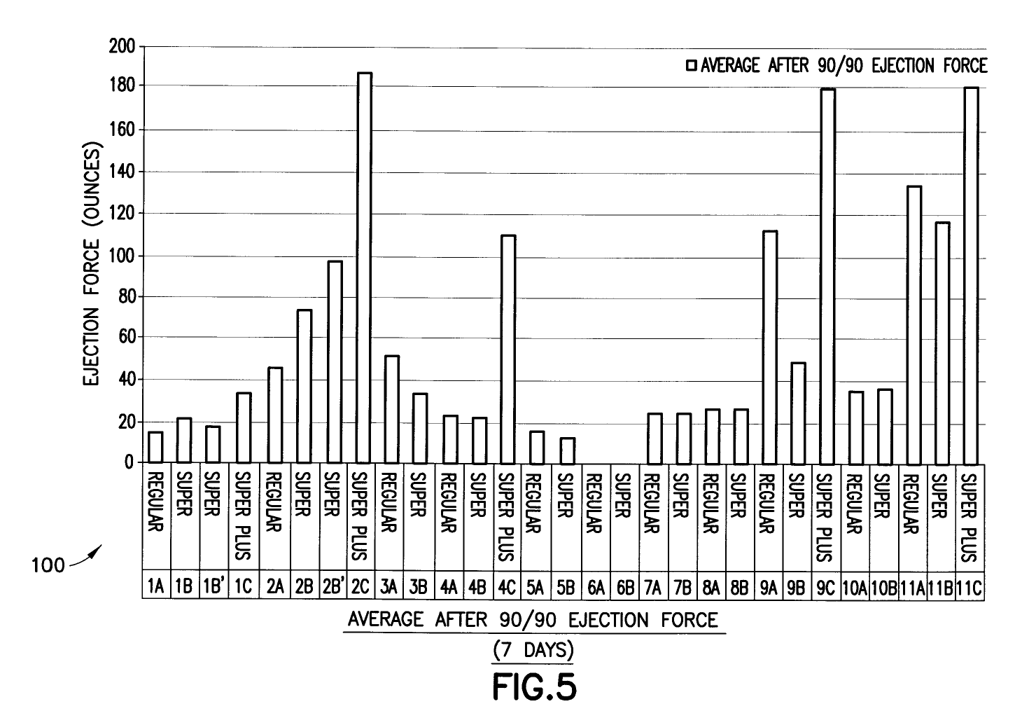

In one embodiment, the package material 10 includes a feature 50 such as, for example, a notch or plurality of aligned perforations, located at one of its ends (e.g., at end 10c) to facilitate opening the tampon package 18 and retrieving the tampon assembly 12 stored therein. As shown in FIGS. 2 and 3, the feature 50 extends from an outside edge of the package material 10 (e.g., end 10c) to a point in close proximity to the perimeter seal 20. The tampon package 18 is opened by exerting force on the package material 10 about the feature 50 to tear the material 10 across the perimeter seam 20 (e.g., seams 22 and 24 or seams 34 and 44). As shown in FIG. 4 and as described above, the feature 50 may also be comprised of a printed pattern of a notch 51 with a cut (e.g., slit) 52 in a portion of the notch 51 such as through a middle of the notch 51. At least one perceived advantage of the printed notch 51 is seen to include the elimination of build up chad waste from cut out portions of the package material 10 that may accumulate on a machine during manufacture. As can be appreciated, such a build up of waste is at least a nuisance and at worst may result in damage to the machine.

In one embodiment, to even further minimize a chance of in-package blooming, the tampon assembly 12 is sealed within the tampon package 18 in a humidity-controlled environment so that any air sealed within the internal chamber 14 contains only minimal, if any, moisture. Alternatively or additionally, the sealing operation may include vacuum packing and/or sterilizing processes such that substantially all air, moisture and contaminates are evacuated from the internal chamber 14 prior to sealing the chamber 14.

While the sealing process above describes steps to minimize, if not eliminate, the opportunity for moisture to penetrate the tampon package 18, it should also be appreciated that the tampon package 18 acts to inhibit contaminates such as, for example, bacteria, dirt and other undesirable materials for entering the internal chamber 14 and compromising sanitary conditions of the tampon assembly 12 and, in particular, the tampon pledget.

As described herein, the tampon package 18 is seen to substantially reduce, if not eliminate, premature tampon pledget blooming and product contamination, resulting in marked improvement over conventional tampon packaging, and allowing stability (at designed levels) in ejection forces required to expel the tampon pledget from the applicator barrel. Another advantage of the present invention is seen to include providing a portable and hygienic tampon package, which is not seen in the prior art.

Some of the advantages of the tampon package 18 constructed and operating in accordance with the present invention are demonstrated by the following studies of existing tampon packages as well as comparisons of the inventive tampon packages to these conventional packages.

For testing purposes, aging is often simulated by purposefully subjecting tampon assemblies to specific conditions of temperature and humidity for specified periods of time. It has been found that aging, particularly aging under humid ambient conditions, can cause an increase in the ejection force needed to dispense the tampon from the applicator barrel.

### EXAMPLE 1

In a first example, results 100 of which are illustrated in FIG. 5, a variety of conventional un-wrapped tampon assemblies were subjected to an aging cycle of ninety degrees Fahrenheit (90° F.) and ninety percent (90%) relative humidity for seven (7) days. At the end of the aging cycle, the tampon assemblies were tested to determine the force required to eject the tampon pledgets from corresponding applicator barrels. The results 100 of the test, indicating required ejection force, ranged from about forty to about one hundred eighty ounces (40 to 180 oz.). As shown, there was a large increase in ejection force of the aged tampon assemblies versus new, unexposed tampon assemblies, for which an ejection force of in a range of ten to sixty ounces (10 to 60 oz.) is typically preferred.

Evaluation of the results 100 of the aging cycle depicted in FIG. 5 indicates that tampon pledgets bloom (e.g., radially expand) prematurely in the applicator barrels as a result of the absorption of moisture typically found in hot and humid environments. For example, a sample, shown generally at 110, of tampon pledgets rated “regular” absorbency (discussed below in Examples 2-5) had a post-aging ejection force of about 45 ounces. Accordingly, the results 100 demonstrate the undesirable effect that aging and, in particular, of aging in hot and humid conditions, can have on tampon assemblies that are not adequately protected from such environments.

Having demonstrated the need for protecting tampon assemblies subject to such environmental conditions, the inventors conducted further tests seeking to evaluate the effectiveness of wrapping tampon assemblies to mitigate the affects of aging.

A first set of tampon assemblies including tampon pledget rated “regular” absorbency were sealed in production wrappers. As used herein, a production wrapper is comprised of a co-extruded laminate film that is 1.0 mil thick and contains a polypropylene (PP) base and an ethylene vinyl acetate (EVA) sealant. The first tampon assembly packages were subjected to an aging cycle of three (3) days at seventy-eight degrees Fahrenheit (78° F.) and seventy-five percent (75%) relative humidity (RH). After this aging cycle, the ejection forces of the first set of tampon assemblies were measured. In addition, a syngyna absorbency test was conducted as described by the U.S. Food and Drug Administration (FDA) testing method (21 CFR 801.430(f)(2)). The syngyna absorbency (e.g., the total absorbency of the tampon at the point of leakage) was measured, as is illustrated in Table 1A below in an “Absorbency” (grams) column. It is important to note that the total absorbencies need to be in parity in order to evaluate the ejection force on a level playing field, otherwise a lower total absorbency pledget would obviously have a lower ejection force at the same absorbent density. The observed results are tabulated in Table 1A, as follows.

TABLE 1A
Production Wrap
Wrap Test “Regular” Absorbency Tampons
Conditions: 78° F./75% RH, in chamber for 3 days
Ejection Forces and Absorbency

| | Ejection | Dry | Wet | | |
|---|---|---|---|---|---|
| | Force | Weight | Weight | Absorbency | Gram per gram |
| | (ounces) | (grams) | (grams) | (grams) | absorbency Gm |
| Number of | 8 | 3 | 3 | 3 | 3 |
| Samples | | | | | |
| Average | 24.31 | 1.75 | 9.61 | 7.86 | 4.55 |
| Std. Dev. | 2.7358 | 0.0505 | 0.2108 | 0.2597 | 0.2792 |
| Maximum | 27.79 | 1.80 | 9.78 | 8.09 | 4.82 |
| Result | | | | | |
| Minimum | 19.95 | 1.70 | 9.38 | 7.58 | 4.26 |
| Result | | | | | |

As illustrated in Table 1A, the average ejection force of the first set of tampon assembly packages was 24.31 ounces, and the total absorbency of the tampons was about 7.86 grams.

A second set of tampon assemblies including tampon pledgets rated “regular” absorbency were placed in wrappers comprising a PP-EVA water barrier material, as described herein, where the EVA was disposed inside the PP, and then sealed with a continuous perimeter seal to provide air-tight, tampon assembly packages. The second set of tampon assembly packages were subjected to a similar aging process as outlined for the first set of tampon assemblies, e.g., an aging cycle of three (3) days at seventy-eight degrees Fahrenheit (78° F.) and seventy-five percent (75%) relative humidity (RH). After this aging cycle, the ejection forces and other measurements were made as previously described. The results are set forth in the following Table 1B.

TABLE 1B
Air-Tight, Water Barrier Wrapper
“Regular” Absorbency Tampons
Conditions: 78° F./75% RH, in chamber for 3 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 8 | 3 | 3 | 3 | 3 |
| of Samples | | | | | |
| Average | 12.72 | 1.61 | 9.45 | 7.84 | 4.68 |
| Std. Dev. | 3.0169 | 0.0511 | 0.3451 | 0.3193 | 0.1818 |
| Maximum | 19.27 | 1.67 | 9.73 | 8.13 | 4.89 |
| Result | | | | | |
| Minimum | 10.05 | 1.57 | 9.06 | 7.50 | 4.55 |
| Result | | | | | |

As illustrated in Table 1B, the average ejection force of the second set of tampon assembly packages was 12.72 ounces, and the total absorbency of the tampons was about 7.84 grams.

By comparing the data of Tables 1A and 1B, the average ejection force of tampon assemblies sealed in the air-tight, water barrier wrapper was significantly less than the average ejection force of the tampon assemblies wrapped in the production wrapper, e.g., 12.72 ounces versus 21.34 ounces, while the total absorbency was similar, 7.84 grams versus 7.86 grams. These first set of exemplary results confirm the inventors' discovery that tampon packaging having a continuous perimeter seal to enclose the tampon assembly within an air, moisture and contaminate resistance wrapper minimizes premature tampon pledget blooming and thus provides for a stable ejection force regardless of the environment under which tampon packages undergo aging.

### EXAMPLE 2

In a second exemplary analysis, the inventors wrapped a third set of tampon assemblies including tampon pledgets rated “regular” absorbency in wrappers comprising the production wrap of Example 1. In this analysis, the tampon assemblies were exposed to a longer period of aging. For example, the third tampon assembly packages were subjected to an aging cycle of seven (7) days at seventy-eight degrees Fahrenheit (78° F.) and seventy-five percent (75%) relative humidity (RH). After this prolonged aging cycle, the ejection forces of the third tampon assemblies and other measurements were made, as described above. The results are set forth in the following Table 2A.

TABLE 2A
Production Wrap
“Regular” Absorbency Tampons
Conditions: 78° F./75% RH, in chamber for 7 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 8 | 3 | 3 | 3 | 3 |
| of Samples | | | | | |
| Average | 26.47 | 1.84 | 9.84 | 8.00 | 3.74 |
| Std. Dev. | 1.5697 | 0.0723 | 0.4014 | 0.3292 | 0.0112 |
| Maximum | 29.04 | 1.89 | 10.11 | 8.23 | 3.75 |
| Result | | | | | |
| Minimum | 23.80 | 1.76 | 9.38 | 7.62 | 3.73 |
| Result | | | | | |

As illustrated in Table 2A, the average ejection force of the third set of tampon assembly packages was 26.47 ounces, and the total absorbency of the tampons was about 8.00 grams.

A fourth set of tampon assemblies including tampon pledgets rated “regular” absorbency were sealed in wrappers comprising a PP-EVA water barrier material, as described in Example 1 and sealed with a continuous perimeter seal to provide air-tight, tampon assembly packages. The fourth set of tampon assembly packages were also subjected to the prolonged aging cycle, e.g., seven (7) days at seventy-eight degrees Fahrenheit (78° F.) and seventy-five percent (75%) relative humidity (RH). After this aging cycle, the ejection forces of the fourth tampon assemblies and other measurements were made, as described above. The results are set forth in the following Table 2B.

TABLE 2B
Air-Tight, Water Barrier Wrapper
“Regular” Absorbency Tampons
Conditions: 78° F./75% RH, in chamber for 7 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 7 | 3 | 3 | 3 | 3 |
| of Samples | | | | | |
| Average | 13.05 | 1.68 | 9.66 | 7.98 | 4.08 |
| Std. Dev. | 1.0690 | 0.0777 | 0.3590 | 0.2813 | 0.0437 |
| Maximum | 14.22 | 1.77 | 10.07 | 8.30 | 4.11 |
| Result | | | | | |
| Minimum | 11.27 | 1.62 | 9.38 | 7.76 | 4.03 |
| Result | | | | | |

As illustrated in Table 2B, the average ejection force of the fourth set of tampon assembly packages was 13.05 ounces, and the total absorbency of the tampons was about 7.98 grams.

By comparing the data of Tables 2A and 2B, the average ejection force of tampon assemblies sealed in the air-tight, water barrier wrapper was significantly less than the average ejection force of the tampon assemblies wrapped in the production wrapper, e.g., 13.05 ounces versus 26.47 ounces, while the total absorbency was similar, 7.98 grams versus 8.00 grams. These exemplary results once again confirm the inventors' discovery, albeit for a longer period of exposure, that tampon packaging having a continuous perimeter seal to enclose the tampon assembly within an air, moisture and contaminate resistance wrapper minimizes premature tampon pledget blooming and thus provides for a stable ejection force regardless of the environment under which tampon packages undergo aging.

### EXAMPLE 3

A fifth set of tampon assemblies including tampon pledgets rated “regular” absorbency were placed in production wrappers, as described in Example 1. The fifth set of tampon packages were then subjected to an aging cycle of seven (7) days at ninety degrees Fahrenheit (90° F.) and ninety percent (90%) relative humidity (RH). After this prolonged and more humid aging cycle, the ejection forces of the fifth set of tampon assemblies and other measurements were made, as described above. The results are set forth in the following Table 3A.

TABLE 3A
Production Wrapper
“Regular” Absorbency Tampons
Conditions: 90° F./90% RH, in chamber for 7 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 8 | 3 | 3 | 3 | 3 |
| of Samples | | | | | |
| Average | 36.06 | 1.86 | 9.57 | 7.71 | 3.57 |
| Std. Dev. | 3.4638 | 0.0430 | 0.1414 | 0.0988 | 0.0382 |
| Maximum | 42.41 | 1.90 | 9.70 | 7.80 | 3.60 |
| Result | | | | | |
| Minimum | 32.27 | 1.82 | 9.42 | 7.61 | 3.53 |
| Result | | | | | |

As illustrated in Table 3A, the average ejection force of the fifth set of tampon assembly packages was 36.06 ounces, and the total absorbency of the tampons was about 7.71 grams.

A sixth set of tampon assemblies including tampon pledgets rated “regular” absorbency were sealed in wrappers comprising a PP-EVA water barrier material, as described in Example 1, and sealed with a continuous perimeter seal to provide air-tight, tampon assembly packages. The sixth set of tampon assembly packages was also subjected to the prolonged and higher humidity aging cycle, e.g., seven (7) days at ninety degrees Fahrenheit (90° F.) and ninety percent (90%) relative humidity (RH). After this aging cycle, the ejection forces of the sixth tampon assemblies and other measurements were made, as described above. The results are set forth in the following Table 3B.

TABLE 3B
Air-Tight, Water Barrier Wrapper
“Regular” Absorbency Tampons
Conditions: 90° F./90% RH, in chamber for 7 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 7 | 3 | 3 | 3 | 3 |
| of Samples | | | | | |
| Average | 16.33 | 1.75 | 9.44 | 7.69 | 3.77 |
| Std. Dev. | 1.8083 | 0.0993 | 0.5099 | 0.4123 | 0.0434 |
| Maximum | 18.81 | 1.86 | 9.97 | 8.11 | 3.82 |
| Result | | | | | |
| Minimum | 14.13 | 1.67 | 8.96 | 7.29 | 3.74 |
| Result | | | | | |

As illustrated in Table 3B, the average ejection force of the sixth set of tampon assembly packages was 16.33 ounces, and the total absorbency of the tampons was about 7.69 grams.

By comparing the data of Tables 3A and 3B, the average ejection force of tampon assemblies sealed in the air-tight, water barrier wrapper was significantly less than the average ejection force of the tampon assemblies wrapped in the production wrapper, e.g., 16.33 ounces versus 36.06 ounces, while the total absorbency of the tampons was similar, 7.69 grams versus 7.71 grams. Once again, these results confirm the inventors' discovery that, even for longer periods of exposure and higher humidity, tampon packaging having a continuous perimeter seal to enclose the tampon assembly within an air, moisture and contaminate resistance wrapper minimizes premature tampon pledget blooming and thus provides for a stable ejection force regardless of the environment under which tampon packages undergo aging.

### EXAMPLE 4

A seventh set of tampon assemblies including tampon pledgets rated “regular” absorbency were placed in production wrappers, as described in Example 1. The seventh set of tampon packages were then subjected to an aging cycle of three (3) days at ninety degrees Fahrenheit (90° F.) and ninety percent (90%) relative humidity (RH). After this shorter and more humid aging cycle, the ejection forces of the seventh set of tampon assemblies and other measurements were made, as described above. The results are set forth in the following Table 4A.

TABLE 4A
Production Wrapper
“Regular” Absorbency Tampons
Conditions: 90° F./90% RH, in chamber for 3 days
Ejection Forces and Absorbency

| | Ejection | Dry | Wet | | Gram per |
|---|---|---|---|---|---|
| | Force | Weight | Weight | Absorbency | gram |
| | (ounces) | (grams) | (grams) | (grams) | absorbency |
| Number | 8 | 3 | 3 | 3 | 3 |
| of Samples | | | | | |
| Average | 31.38 | 1.89 | 9.97 | 8.08 | 4.43 |
| Std. Dev. | 5.4055 | 0.0420 | 0.0681 | 0.0405 | 0.0928 |
| Maximum | 37.63 | 1.94 | 10.04 | 8.11 | 4.53 |
| Result | | | | | |
| Minimum | 20.90 | 1.85 | 9.91 | 8.03 | 4.35 |
| Result | | | | | |

As illustrated in Table 4A, the average ejection force of the seventh set of tampon assembly packages was 31.38 ounces, and the total absorbency of the tampons was about 8.08 grams.

An eighth set of tampon assemblies including tampon pledgets rated “regular” absorbency were sealed in wrappers comprising a PP-EVA water barrier material, as described in Example 1, and sealed with a continuous perimeter seal to provide air-tight, tampon assembly packages. The eighth set of tampon assembly packages were also subjected to the higher humidity aging cycle, e.g., three (3) days at ninety degrees Fahrenheit (90° F.) and ninety percent (90%) relative humidity (RH). After this aging cycle, the ejection forces of the eighth tampon assemblies and other measurements were made, as described above. The results are set forth in the following Table 4B.

TABLE 4B
Air-Tight, Water Barrier Wrapper
“Regular” Absorbency Tampons
Conditions: 90° F./90% RH, in chamber for 3 days
Ejection Forces and Absorbency

| | Ejection | Dry | Wet | | Gram |
|---|---|---|---|---|---|
| | Force | Weight | Weight | Absorbency | per gram |
| | (ounces) | (grams) | (grams) | (grams) | absorbency |
| Number | 8 | 3 | 3 | 3 | 3 |
| of Samples | | | | | |
| Average | 15.16 | 1.62 | 9.40 | 7.78 | 4.69 |
| Std. Dev. | 2.0910 | 0.1273 | 0.4545 | 0.3297 | 0.1880 |
| Maximum | 18.04 | 1.74 | 9.85 | 8.12 | 4.91 |
| Result | | | | | |
| Minimum | 10.79 | 1.49 | 8.95 | 7.46 | 4.57 |
| Result | | | | | |

As illustrated in Table 4B, the average ejection force of the eighth set of tampon assembly packages was 15.16 ounces, and the total absorbency of the tampons was about 7.78 grams.

By comparing the data of Tables 4A and 4B, the average ejection force of tampon assemblies sealed in the air-tight, water barrier wrapper was significantly less than the average ejection force of the tampon assemblies wrapped in the production wrapper, e.g., 15.16 ounces versus 31.38 ounces, while the total absorbency of the tampons was fairly similar, 7.78 grams versus 8.08 grams.

The foregoing data illustrates that tampon assemblies wrapped within tampon packaging having a continuous perimeter seal to enclose the tampon assembly within an air, moisture and contaminate resistance wrapper, minimizes premature tampon pledget blooming and thus provides for a stable ejection force regardless of the environment under which tampon packages undergo aging. As such, the adverse effects of aging on the ejection force of the tampon assembly are substantially reduced, even relative to aging that includes exposure to ambient humidity. This improvement is achieved without the need to increase the compression of the tampon pledget, which is advantageous because with increased compression, the fibers of the tampon (which typically comprise rayon) can become overly compacted, leading to a reduced rate of absorbency. Accordingly, this invention provides resistance to increased ejection force without a loss of absorbency. In addition to reducing the ejection force of the tampon, the air-tight water barrier wrapper provides the user with a well-sealed package that decreases a chance of the package opening (e.g., opening prior to use) and, thus, becoming contaminated. Also, the wrapper comprising a PP-EVA water barrier material provides the consumer with a soft and quiet (when opened) package. As can be appreciated by those skilled in the art, the combination of an improved seal and quieter opening package are important benefits provided to consumer that are not seen in prior art tampon packages.

The terms “first,” “second,” and the like, herein do not denote any order, quantity, or importance, but rather are used to distinguish one element from another. In addition, the terms “a” and “an” herein do not denote a limitation of quantity, but rather denote the presence of at least one of the referenced item.

Although the invention has been described with reference to particular embodiments thereof, it will be understood by one of ordinary skill in the art, upon a reading and understanding of the foregoing disclosure, that numerous variations and alterations to the disclosed embodiments will fall within the spirit and scope of this invention and of the appended claims.

What is claimed is:

1. A tampon package, comprising:
a tampon assembly including a tampon pledget disposed within an applicator barrel; and
package material having a perimeter, the package material being continuously sealed about the perimeter to form an internal air-tight chamber for retaining the tampon assembly therein.

2. The tampon package of claim 1, wherein the package material is comprised of a water-barrier material.

3. The tampon package of claim 2 wherein the water-barrier material is comprised of a laminate including a thermoplastic material.

4. The tampon package of claim 2, wherein the water-barrier material is comprised of a laminate including a layer of a polyolefin and layer of an ethylene vinyl acetate material.

5. The tampon package of claim 4, wherein the layer of ethylene vinyl acetate material is inside the layer of polyolefin material.

6. The tampon package of claim 1, wherein the continuous perimeter seal includes a first unitary perimeter seal.

7. The tampon package of claim 1, wherein the continuous perimeter seal includes a first unitary perimeter seal and a second unitary perimeter seal disposed closer to the perimeter of the package material than the first perimeter seal.

8. The tampon package of claim 7, wherein the first perimeter seal and the second perimeter seal are each formed by a single sealing operation.

9. The tampon package of claim 8, wherein the first perimeter seal and the second perimeter seal are formed at least one of simultaneously and consecutively.

10. The tampon package of claim 1, wherein the continuous perimeter seal includes:
a first end seam formed at a first end of the packaging material;
a second end seam formed at a second end of the packaging material; and
a side seam formed at a side of the packaging material and intersecting the first end seam and the second end seam.

11. The tampon package of claim 10, wherein the first end seam, the second end seam and the side seam form a first group comprising a first continuous perimeter seal, and wherein a second group of a first end seam, a second end seam and a side seam form a second continuous perimeter seal.

12. The tampon package of claim 1, further including a feature disposed in the packaging material to facilitate tearing of the packaging material.

13. The tampon package of claim 12, wherein the feature is comprised of a notch in the packaging material.

14. The tampon package of claim 12, wherein the feature is comprised of a printed notch on the packaging material with a slit located in a portion of the printed notch.

15. The tampon package of claim 1, wherein air, moisture and contaminates are evacuated from the internal chamber prior to forming of the perimeter seal.
